(19) United States
(12) Patent Application Publication
(10) Pub. No.: US 2026/0263353 A1
(43) Pub. Date: Sep. 10, 2026

(54) PROGRAMMABLE DRUG-DEVICE MICRONEEDLE SYSTEM FOR THE DELIVERY AND MONITORING OF DIABETES AND OBESITY MEDICATIONS

(71) Applicant: ALJ Creative Works, LLC, St. Louis, MO (US)

(72) Inventors: Ralph Oiknine, St. Louis, MO (US); Thanh D. Nguyen, South Windsor, CT (US); Raghavan Rajagopalan, St. Peters, MO (US)

(21) Appl. No.: 19/153,321

(22) PCT Filed: Feb. 7, 2024
(86) PCT No.: PCT/US2024/014724

§ 371 Date: Aug. 1, 2025

Related U.S. Application Data
(60) Provisional application No. 63/490,730, filed on Mar. 16, 2023.

(51) Int. Cl. A61K 9/00; A61K 9/70; A61K 38/22; A61K 38/26; A61K 45/06; A61K 47/54; A61K 49/00; C07K 16/28

(52) U.S. Cl. CPC A61K 9/0021; A61K 9/7084; A61K 38/22; A61K 38/26; A61K 45/06; A61K 47/543; A61K 49/006; C07K 16/2869

(57) ABSTRACT

This invention relates to drug-device core-shell microneedle devices containing anti-diabetes and anti-obesity medications, and transdermal delivery of said medications. The microneedle drug-device system comprises: (a) a two-dimensional array of conical bilayer containing an inner layer with the base diameter ranging from about 100 μm to about 500 μm and the height ranging from about 100 μm about 1200 μm capable of accommodating a known amount of anti-diabetes and/or anti-obesity medications, and a suitable, photostable chromophore or a fluorophore whose absorption and/or emission occurring in the range of 400-900 nm; (b) a larger outer layer that adheres to and encapsulates the inner layer, and protects the inner layer, and (c) a two-dimensional array of cylindrical ‘cap layer’ that aligns with and adheres to the basal surface of core shell bilayer and seals the bilayer to prevent leakage of said medication(s). The apical (i.e., sharp) ends of the conical bilayer array inserts into the skin and are programmed to allow the said medications to effuse out of the bilayer and into the skin in a controlled fashion and to be monitored by photodetection devices.

This application claims benefit of priority based on provisional application No. 63/490,730 filed on Mar. 16, 2023, and said application is incorporated herein by reference in its entirety.

### CROSS-REFERENCE TO RELATED APPLICATIONS

Not Applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

ALJ Creative Works, LLC and University of Connecticut.

### INCORPORATION-BY-REFERENCE OF MATERIAL SUBMITTED ON A COMPACT DISC

Not applicable.

### BACKGROUND OF THE INVENTION

### Field of the Invention

This invention relates to the treatment of diabetes, obesity, and related conditions. Particularly, this invention relates to drug-device core-shell microneedle devices containing anti-diabetes and anti-obesity medications, and transdermal delivery of said medications.

### Description of the Related Art

Unless otherwise specified, all technical terms and phrases used herein conform to standard nomenclature established in the respective arts. Various prior art references in the specification are indicated by italicized Arabic numerals in brackets. Full citation corresponding to each reference number is listed at the end of the specification and is herein incorporated by reference in its entirety in order to describe fully and clearly the state of the art to which this invention pertains.

Diabetes and its related complications continue to increase worldwide due to demographic changes, improper dietary control, and other risk factors. Uncontrolled diabetes leads to cardiovascular, renal, cognitive, and neurodegenerative disorders; peripheral and autonomic neuropathy; depression; nonalcoholic steatohepatitis (NASH), erectile dysfunction (ED), arthritis and bone loss among other complications. Some of the most effective medications for the treatment of diabetes include peptide hormones that are involved in blood glucose regulation and appetite regulation. Peptide hormones are produced in glands and in several other tissues including the stomach, the intestine, the brain, and carry information from one tissue through the blood to another. Peptide hormones that are involved in blood glucose regulation include insulin, secreted by pancreatic beta cells; amylin, co-secreted with insulin from pancreatic beta cells; glucagon, secreted by the pancreatic alpha cells; incretin hormones such as glucagon-like-peptide-1 (GLP-1), secreted by the intestinal L cells; and glucose dependent insulinotropic polypeptide (GIP), secreted by the intestinal K cells. In addition to peptide hormones involved in glucose regulation, other peptide hormones are involved in appetite regulation and include Ghrelin, Leptin, Cholecystokinin (CCK), Oxyntomodulin (OXM), Peptide YY (PYY), Pancreatic Polypeptide (PP), and the like.

GLP-1 receptor (GLP-1R) belongs to Family B1 of the seven transmembrane G-protein coupled receptors (GPCRs). Its natural agonist ligands are glucagon and the related peptide hormone, GLP-1, which is involved in glucose homeostasis. The activation of GLP-1R in the plasma membrane of pancreatic cells stimulates glucose dependent insulin secretion, reduce body weight, and over time, benefits cardiovascular health [1-3]. GLP-1R receptor agonists (GLP-1RA) represent one the most effective FDA-approved drug classes for the treatment of diabetes and obesity, and are currently administered as injectables once daily (Liraglutide), twice daily (Exenatide), or once weekly (Semaglutide, Dulaglutide and Exenatide LAR) [4]. Semaglutide is now available as the first oral GLP-1RA medication approved by the FDA for the treatment of type-2 diabetes. Other oral GLP-1RA drugs are under development. In addition to the approved GLP-1RA medications above, several attempts have been made to combine two or even three biologically active peptides into one molecule during the last decade. A dual agonist GLP-1RA/peptide combination such as GLP-1RA/GIP (Tirzepatide) has been approved by the FDA. Other dual combinations such as GLP-1RA/Glucagon (Cotadutide), and triple agonist combination such as GLP-1RA/GIP/Glucagon, are currently under preclinical and clinical investigation [5]. Further, an amylin analog, Cagrilintide, is under development alone and in combination with Semaglutide [6,7].

GIP alone has not been marketed as a drug to date. Despite being a potent incretin hormone and powerfully stimulating insulin secretion during meal intake, it also contributes to the development of body fat by enhancing deposition of fat in the adipose tissues [8]. Furthermore, GIP loses its insulinotropic effect in diabetic individuals and may be diabetogenic because it retains a stimulatory effect on glucagon secretion [8]. However, the GIP receptor (GIPR) knockout mice develop resistance toward diet induced obesity, and people with inactivating mutations of GIP receptor show reduced body weight [9]. Recently, a composition comprising a monoclonal antibody directed against the human and/or the murine GIPR conjugated with GLP-1RA was shown to reduce body weight and improve metabolic parameters in mice and monkeys [10]. In addition, said compositions have a long duration of action that renders them suitable for at least weekly use. The findings are of great interest in view of the current development of pharmaceutical agents based on GLP-1RA and GIP. In addition to incretin hormone combinations, given the association with diabetes and atherosclerotic cardiovascular disease, a peptide combination with a antihyperlipidemic molecule GLP-1/PCSK9 antibody has been described and is being developed [11].

Currently, the treatment of obesity involves both medical therapy and lifestyle interventions. Specific GLP-1RAs such as once daily, high dose liraglutide (Saxenda) and once weekly high dose Semaglutide (Wegovy) have been approved for the treatment of obesity. Further, specific GLP1-RA's such as semaglutide and dulaglutide are FDA approved for prevention of cardiovascular disease [12, 13]. Dulaglutide is also showing promise for the prevention of progression of chronic kidney disease [14] and erectile dysfunction [15]. Furthermore, pramlintide, the analogue of peptide hormone, amylin is FDA-approved not only for the treatment of type 1 and type 2 diabetes but is also being investigated for weight loss, either alone or in combination with other peptide hormones such as GLP-1RA [16, 17]. Preliminary efficacy data with double and triple peptide combinations such as GLP-1RA/GIP, GLP-1RA/Glucagon, GLP-1RA/GIP/Glucagon combinations are very promising, and these combinations are currently under FDA review for the treatment of diabetes, obesity [18-20], and NASH [21]. Type 2 diabetes has been identified as a major risk factor for the development of Alzheimer's disease. Recent evidence indicates that short amylin receptor antagonist peptides improved memory deficits in an Alzheimer's mouse model [22]. Further, peptides such as Lixisenatide and Pramlintide were found to have neuroprotective effects on Alzheimer's disease pathogenesis and cognition [23, 24]. When medical therapy and lifestyle intervention fail, bariatric surgery is indicated for individuals with a body mass index (BMI) over or equal to 40 or more than 100 pounds overweight without coexisting medical problems. Bariatric surgery is also indicated for individuals with a BMI over or equal to 35 and at least one or more obesity related co-morbid conditions such as type 2 diabetes, hypertension, hyperlipidemia, sleep apnea and other respiratory disorders, non-alcoholic fatty liver disease, debilitating arthritis or considerably impaired quality of life, GERD, venous stasis disease, and cardiovascular disorders. Other candidates for bariatric surgery include patients with a BMI of 30 to 34.9 with diabetes or metabolic syndrome.

Microneedle patches incorporating small molecule drugs or biologics have advantages over conventional hypodermic needles for delivery of these medications [25]. Insulin is very effective in lowering blood glucose levels, and recent advances in insulin therapy resulted in once weekly basal insulin such as Insulin ICODEC, which allows for once weekly dosing at clinically relevant doses. Further, Insulin ICODEC was shown to be safe and well-tolerated with favorable half-life of about 196 hours [26]. Zhou et al. [27] provide a comprehensive review of delivery of insulin using microneedles. Many microneedle systems for insulin delivery are under clinical trials, but none thus far have been introduced into the market.

Design and fabrication of microneedle patch system specific system for transdermal delivery of specific medication depends on many physicochemical properties of both the patch system and the medication. These properties include molecular structure, topology, charge, membrane permeability (which is related to lipophilicity of said medication), and adhesion property between of the medication and the microneedle patch. Most of the current microneedle platforms deliver drugs and biologics either via bolus or sustained release without any modulation. Other technologies employing hollow microneedles do provide a micro-infusing system, but require an external drug reservoir placed on the skin or on portable devices. Recently developed microparticle system, ‘StampED Assembly of Polymer Layers (SEAL),’ has been shown to deliver vaccines in a singly-administered multi-pulse delivery of vaccines [28,29]. However, this SEAL system relies on a tedious, inefficient manual process to remove scum/residual layers of polymer after molding steps and a slow process to fill drugs sequentially (i.e., one-by-one) into polymeric carriers. This filling process is beset with instability of off-target jetting, and severely suffers from the ‘coffee-ring’ effect that results in drugs being deposited on the edges of the drug micro-cores thereby preventing a maximal loading efficiency. Further, the SEAL system has been shown to create different polymeric layers that often results in the formation of a thick scum residual layer which requires a manual, inconsistent and slow removal process using scotch-tape and oxygen plasma. One of the earliest and most commonly used and effective treatment for diabetes is injectable insulin or insulin derivatives.

The new microneedle patch system conceived and developed by us [28], and which are incorporated herein by reference in its entirety, overcomes the aforementioned problems, and enables the delivery of drugs and biologics in a programmable, pulsatile manner.

### SUMMARY OF THE INVENTION

The present application particularly relates to said microneedle patch system for the delivery of anti-diabetes and anti-obesity medications. We [28, 29] and others [30] have provided general method for the design and fabrication of microneedle patches. It should be emphasized, however, that the design and development of microneedle system incorporating various drugs and biologics depends on the molecular weight, size, in vitro and in vivo stability, adhesion properties, release kinetics from microneedle, pharmacokinetics, membrane permeability, and the like that are not accurately predictable from the prior art. Development of optimal microneedle delivery systems for anti-diabetes and anti-obesity medications require considerable effort and cannot be ascertained from any of the prior art disclosures without undue experimentation.

Accordingly, the present invention particularly relates to a drug/device combination for a controlled, singly-administered, and therapeutically effective transdermal delivery of medications for the treatment of diabetes, obesity, and diabetes/obesity associated conditions including, but not limited to vascular disease (small and large vessel disease), NASH, hyperlipidemia, peripheral neuropathy, chronic kidney disease, erectile dysfunction, depression, or dementia. Particularly, this invention relates to a specialized, clinically acceptable biodegradable core-shell microneedle drug-device ensemble for programmable pulsatile delivery of GLP-1RA agents such as Exenatide, Semaglutide, Liraglutide, Dulaglutide, Lixisenatide, albiglutide, and the like, alone or in combination with peptide hormones such as glucagon, GIP, amylin, leptin, CCK, OXM, PYY, PP and the like, and the detection and tracking of said GLP-1RA agents and peptide hormones using non-invasive photophysical method. The present invention also relates to the transdermal, pulsatile delivery of therapeutically effective peptide hormones alone or in combination, with GLP-1RA, for the treatment of diabetes, obesity, and diabetes/obesity associated conditions.

The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated, the severity of the disorder; activity of the specific compound employed; the specific composition employed, age, body weight, general health, sex, diet of the patient; the time of administration, route of administration, and rate of excretion of the specific compound employed, and the duration of the treatment.

The microneedle drug-device system of the present invention comprises following components (FIGS. 1 and 2): (a) a two-dimensional array of conical bilayer (referred to a ‘core-shell’) containing an inner layer with the base diameter ranging from about 100 μm to about 500 μm and the height ranging from about 100 μm about 1200 μm capable of accommodating a known amount of anti-diabetes and/or anti-obesity medications, (b) a larger outer layer that adheres to and encapsulates the inner layer, and protects the inner layer, and (c) a two-dimensional array of cylindrical ‘cap layer’ that aligns with and adheres to the basal surface of core shell bilayer and seals the bilayer to prevent leakage of said medication(s). The apical (i.e., sharp) ends of the conical bilayer array inserts into the skin and are programmed to allow the said medications to effuse out of the bilayer and into the skin in a controlled fashion (FIG. 3).

The core-shell microneedle drug-device system of the present invention further comprises an inclusion of a suitable, photostable light absorbing and/or emitting molecule (i.e., chromophore or a fluorophore respectively) in the inner layer along with the anti-diabetes or anti-obesity medications whose absorption and/or emission occurs in the visible (400-650 nm) or near-infrared (NIR) region (650-900 nm) of the electromagnetic spectrum. Said chromophores or fluorophores (hereinafter collectively referred to as ‘dyes’) and their diffusion and effusion properties can be tuned to mimic the clearance rate of anti-diabetes or anti-obesity medications such that the concentration of these medications over time can be accurately measured using photonic devices similar to the current pulse oximeter that measures blood oxygen levels. As diffusion and effusion of molecules is related to their molecular weights, it is reasonable to expect that the dye and the medication having similar molecular weight should have similar transport properties. With the exception of leptin, dulaglutide, and albiglutide, whose molecular weights are about 16 kDa, 60 kDa, and 73 kDa respectively, the molecular weights of all other known medications listed in paragraph [0003] ranges from about 3500 to about 5000 Da (Table 1). Hence, the clearance rate of the dyes can be correlated with the clearance rate of the medications by preparing dye whose molecular weight is similar to those of the medications. This can be

TABLE 1
Molecular Weights of Selected Peptide Hormones

| | MW | | MW | | MW |
|---|---|---|---|---|---|
| PEPTIDE | (Da) | PEPTIDE | (Da) | PEPTIDE | (Da) |
| Ghrelin | 3,314 | CCK | 3,931 | OXM | 4,445 |
| Glucagon | 3,485 | Pramlintide | 3,949 | Lixisenatide | 4,858 |
| hGIP (1-30) | 3,532 | PYY | 4,049 | hGIP (1-42) | 4,983 |
| Cotadutide | 3,728 | Liraglutide | 4,113 | Leptin | 16,000 |
| Semaglutide | 3,751 | Exenatide | 4,186 | Dulaglutide | 59,670 |
| Amylin | 3,903 | PP | 4,200 | Albiglutide | 72,971 |

accomplished by covalently attaching said dyes (whose molecular weights are typically below 500 Da) to a biocompatible, non-photoactive side chains such as polyethylene glycols (PEG's) and the like. Attachment of polyethylene glycol units offer additional advantage of mitigating possible immunoreactivity associated with other types of large side chain attachments.

The core-shell microneedle drug-device system of the present invention would be fully inserted inside the skin and then release the anti-diabetes and/or anti-obesity medications as sharp bursts (boluses) in a predictable, programmed, and controlled manner with a single administration. This one-time application of the microneedle drug-device patch on skin is an easy-to-use, painless method for delivering anti-diabetes and anti-obesity medications repeatedly over a long period of time. The frequency of use of the microneedle patch can vary according to the medical conditions of the individual and can be increased or decreased. The present invention enhances patient compliance and adherence to treatment by setting a one-time dissolvable micro-needle patch administered every week or customized to be administered every 2, 3, 4, or up to 12 weeks or potentially even over 24, 36, 48 or 52 weeks and beyond. The programmable microneedle system eliminates repetitive and painful injections experienced with conventional administrations.

### BRIEF DESCRIPTION OF THE DRAWINGS

FIG. 1. Fabrication of microneedle: hollow outer layer.

FIG. 2. Fabrication of microneedle: drug-filled inner layer.

FIG. 3. Fabrication of microneedle: cap layer.

FIG. 4. Complete exenatide microneedle patch system.

### DETAILED DESCRIPTION

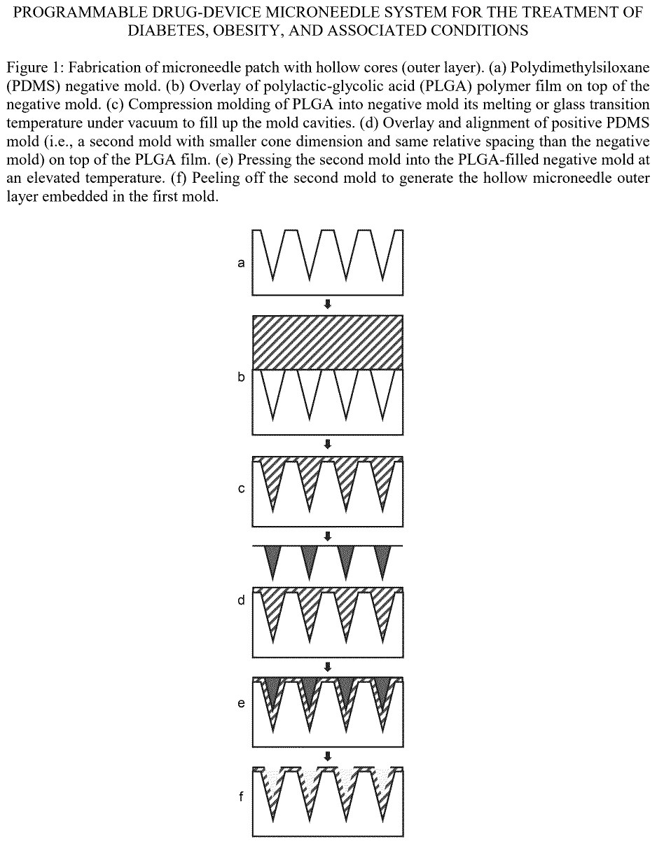

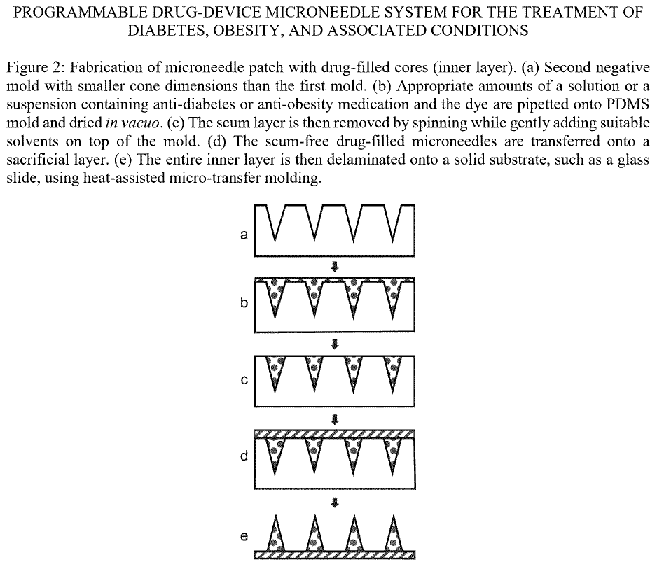

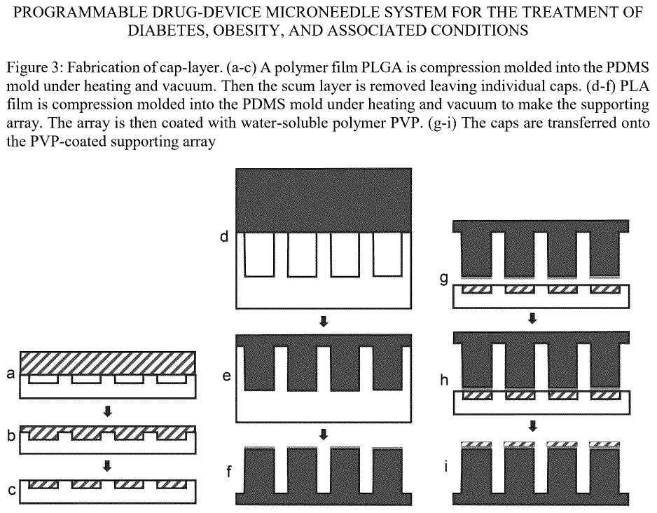

The present invention relates to the controlled, pulsatile, delivery of effective amounts of anti-diabetes and/or anti-obesity peptide hormones via transdermal microneedle system comprising:
(a) a biodegradable polymeric ‘outer layer’ fabricated as a two-dimensional array of plurality of hollow, conical microneedles with the base diameter ranging from about 100 μm to about 500 mm and the height ranging from about 100 μm about 1200 μm that adheres to and fully encapsulating an inner layer (FIG. 1), wherein said biodegradable polymer includes, but not limited to polyglycolic acid (PGA), polylactic acid (PLA), or polylactic-glycolic acid (PLGA); and wherein said outer layer protects the inner layer;
(b) a biodegradable polymeric ‘inner layer’ fabricated as a two-dimensional array of plurality of conical drug-containing microneedles with the base diameter ranging from about 100 μm to about 500 mm and the height ranging from about 100 μm about 1200 μm, wherein said drug-containing microneedles are loaded with anti-diabetes and/or anti-obesity peptide hormones (FIG. 2); a biocompatible dye absorbing and/or emitting in the visible or NIR region; and wherein said biodegradable polymer includes, but not limited to polyglycolic acid (PGA), polylactic acid (PLA), or polylactic-glycolic acid (PLGA); and
(c) a biodegradable polymeric ‘cap layer’ fabricated as a two-dimensional array of plurality of cylindrical discs with the base diameter ranging from about 100 μm to about 500 μm and the height ranging from about 100 μm about 500 μm that aligns with and adheres to the basal surface of said inner and outer layers (FIG. 3), wherein said biodegradable polymer includes, but not limited to polyglycolic acid (PGA), polylactic acid (PLA), or polylactic-glycolic acid (PLGA).

Among the very large classes dyes that are known and can be used in the present invention, pyrazine derivatives are most desirable due to their favorable optical properties and biocompatibility. In fact many pyrazine derivatives are natural products found in vegetables such as bell peppers, green tomatoes, etc, and many of them are used as food flavor agents [31]. Pyrazine derivatives of general Formula 1 where the electron donating group (EDG) and electron withdrawing group (EWG) are positioned anti-symmetrically with respect to the plane intersecting

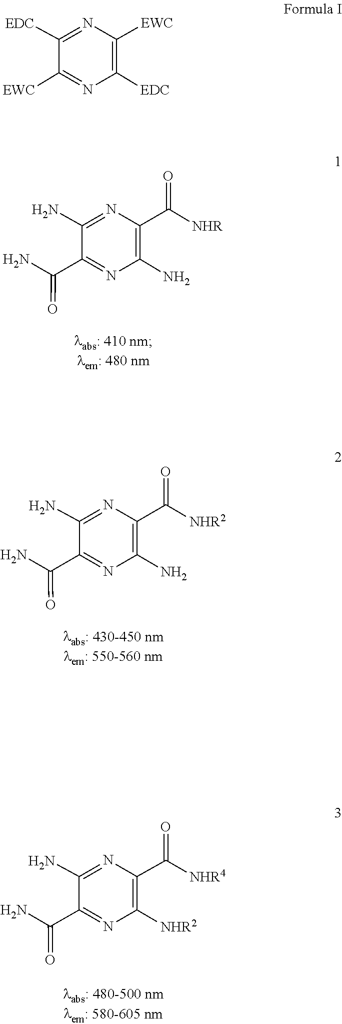

the two nitrogen atoms in the pyrazine ring are one the very few classes of photostable small molecule dyes that absorb and emit in the visible region with a large Stokes shift in the order of ~100 nm and with fluorescence quantum yield of about 0.4 [32], which are exemplified by structures 1-3. Further, a hydrophilic pyrazine derivative containing serine side chains [33, 34] is now undergoing Phase 2 clinical trials for optical monitoring of renal function via a supradermal photonic device that detects fluorescent signals emanating through the skin. U.S. Pat. No. 8,664,392 [35] and 9,283,288 [36] disclose the method of preparing pyrazine dye bioconjugates, which are incorporated herein by reference in their entirety.

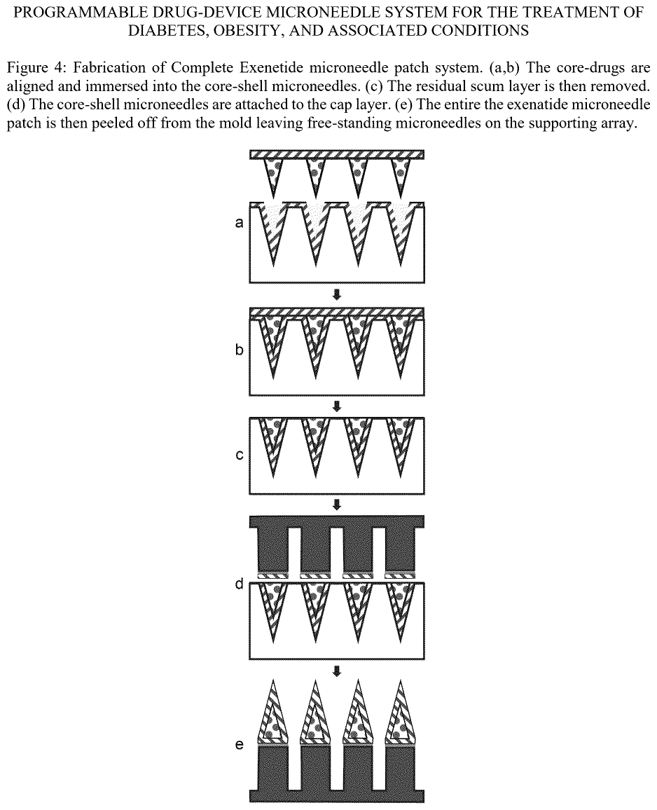

The complete drug-device micro needle ensemble is shown in FIG. 4, and in vitro and in vivo release profile of exenatide- and semaglutide-filled microneedles are shown in FIG. 5 and FIG. 6 respectively.

One embodiment of the present invention is related to the drug-device microneedle patch system wherein said inner layer is filled with a peptide hormone related to diabetes, obesity, and associated comorbidity; and a dye absorbing or emitting light with the wavelength ranging from 400-900 nm.

Another embodiment of the present invention is related to the drug-device microneedle patch system wherein said inner layer is filled with anti-diabetes medication; and a dye absorbing or emitting light with the wavelength ranging from 400-900 nm.

Another embodiment of the present invention is related to the drug-device microneedle patch system wherein said inner layer is filled with anti-obesity medication; and a dye absorbing or emitting light with the wavelength ranging from 400-900 nm.

Another embodiment of the present invention is related to the drug-device microneedle patch system wherein said inner layer is filled with GLP-1RA ligands; and a dye absorbing or emitting light with the wavelength ranging from 400-900 nm.

Another embodiment of the present invention is related to the drug-device microneedle patch system wherein said inner layer is filled with GLP-1RA ligands, including but not limited to exenatide, liraglutide, semaglutide, dulaglutide, lixisenatide, or albiglutide; and a dye absorbing or emitting light with the wavelength ranging from 400-900 nm.

Another embodiment of the present invention is related to the drug-device microneedle patch system wherein said inner layer is filled with anti-obesity medication; and a dye absorbing or emitting light with the wavelength ranging from 400-900 nm.

Another embodiment of the present invention is related to the drug-device microneedle patch system wherein said inner layer is filled with GIP, amylin, leptin, ghrelin, CCK, OXM, PYY, or PP; and a dye absorbing or emitting light with the wavelength ranging from 400-900 nm.

Another embodiment of the present invention is related to the drug-device microneedle patch system wherein said inner layer is filled with GIPR antibody alone or in combination with GLP-1RA ligands; and a dye absorbing or emitting light with the wavelength ranging from 400-900 nm.

Another embodiment of the present invention is related to the drug-device microneedle patch system wherein said inner layer is filled with antihyperlipidemic molecule GLP-1RA/PCSK9 inhibitor; and a dye absorbing or emitting light with the wavelength ranging from 400-900 nm.

Another embodiment of the present invention is related to the drug-device microneedle patch system wherein said inner layer is filled with any combinations of the peptide hormones listed in paragraphs [0025] and [0027]; and a dye absorbing or emitting light with the wavelength ranging from 400-900 nm.

GLP-1RA/GIP and other related combinations, which are single dose injectables, have been shown effective for the treatment of diabetes and obesity. Hence, the microneedle patch of the present invention is purported to release these medications not only on a weekly basis up to 4 weeks (FIG. 6). Said patch is applied to the skin resulting in a weekly or monthly GLP-1RA drug release, and potentially over 12-weeks and beyond resulting in glycemic control and weight loss. The patch is also useful for the treatment of obesity even in non-diabetic individuals as several GLP-1RAs such as semaglutide and liraglutide were approved by the FDA for that purpose. Thus, the drug delivery system according to the present invention provides a one-time administration of several dosages which are programmed to release at predictable time points.

The core-shell microneedle drug/device ensemble of the present invention overcomes the aforementioned SEAL limitations in fabrication, provides a minimally invasive approach, and allows for self-administration. Microneedles are, therefore, an appealing delivery approach to enable one-time, painless, and effective administration of anti-diabetes/anti-obesity medications to replace multiple injections in the conventional process. The microneedles are fabricated from FDA approved materials (e.g., Poly(D,L-lactide-co-glycolide) (PLGA), poly-lactide acid (PLA) that are commonly used for drug delivery, medical devices, etc.

The details of fabrication of core-shell microneedle arrays, and filling of the cores with drugs and vaccines have been reported by us previously [28, 29], and are incorporated by reference herein in their entirety. Fabrication of the three layers of the present invention requires three silicone or polydimethylsiloxane (PDMS) molds to form the cone-shaped microneedles and cylinder-shaped caps.

Briefly, a biodegradable two-dimensional polymer film made PGA, PLA, PLGA, and the like is compression-molded into a pre-fabricated negative mold containing an array of conical cavities. Thereafter, a separate positive two-dimensional hydrophobic PLA mold with a smaller conical dimension but with an identical array than the negative mold is aligned by a custom-built alignment system and impinged into the microneedles entrapped inside the negative PDMS mold at elevated temperature, typically about 50-100° C. This subtractive process creates conical dimple-like structures which serve as the microneedle. After peeling off the PLA mold, the shell with empty outer layer of the present invention containing two-dimensional array of microneedle is obtained and is trapped inside the negative PDMS mold.

A second negative two-dimensional PDMS mold having the same structure and spacing as the outer layer, but slightly smaller cone dimensions, is prepared in the similar manner as first negative PDMS mold. Thereafter, a solution of drug or biomolecules of interest in a solution of aqueous polyvinylpyrrolidone (PVP) prepared, and the entire mixture is solution-casted (i.e. not filled into each micro-well of the mold) onto to the second PDMS mold and allowed to dry to obtain the drug-filled microneedle inner layer. The drugs can also be dissolved inside just a water based solution containing excipients such as trehalose or sucrose which can stabilize the drug and form a solid matrix drug-core after molding process. The micro-molded drug array cores are then aligned and loaded into arrays of empty microneedle outer layer. This single-step and high-throughput loading allows drugs to fill the entire volume of the microneedle cores, thereby significantly increasing the loading consistency and efficiency. Further, this method also does not use any organic solvents or high temperature, thereby providing a gentle process to avoid potential damage to the drug bioactivity. Importantly, this fabrication process introduces a new method for scum removal (i.e. residual layer after each molding) by rapidly spinning PDMS molds while gently adding appropriate solvents on top of the molds to remove hydrophobic polymers or water to remove hydrophilic drugs.

The two-dimensional cap layer was fabricated in the similar manner of compression molding and scum removal described for the outer and inner layers, except that the cavities in negative PDMS mold are cylindrically-shaped. Briefly, a polymer film of PLGA or PLA was compression molded into PDMS mold of the caps and supporting array, respectively. The supporting array was transferred to a glass substrate and further coated with a water-soluble polymer solution of PVP K30 in ethanol (0.2 g/ml), then air-dried for 24 hours. The cap layer is then aligned with the drug-filled inner layer encapsulated by the outer layer and sintered using heat. Finally, the core-shell microneedles are capped and transferred onto a PVP coated PLA supporting array. The supporting array is pre-coated with a water-soluble polymer (PVP) which can be dissolved by local bodily fluid at the site of insertion to release the drugs/vaccines into the skin. The mold entrapping the core-shell microneedles is then peeled off to yield free-standing core-shell microneedles on the supporting array. Specifically, the manufacture of core-shell microneedle drug-device system for anti-diabetes or anti-obesity medications are described below. It is to be understood, however, that the invention is not limited in its application to the details of construction and the arrangement of components set forth in the following description or illustrated in the drawings. As would be apparent to skilled artisans, various modifications in the composition, operation, and method are possible, and are contemplated herein without departing from the concept and scope of the invention as defined in the claims.

### Example 1 (Prophetic)

### Exenatide Core-Shell Microneedle Patch System

Step 1. Fabrication of microneedle outer layer. Poly(D,L-lactide-co-glycolide) (PLGA) (MilliporeSigma and Polysciences, Inc., USA) is dissolved in acetone (20% w/v) and casted as a film on a Teflon-coated petri dish. The film is then lyophilized for complete removal of the organic solvent. A sufficient amount of PLGA film is placed between the PDMS mold and a Teflon film which sat on two glass slides. Then the polymer is compression molded under a binder clip in the vacuum oven at 60° C. for 1-2 hours depending on the polymer properties. To create the core structure, a positive mold made from Poly-lactide acid (PLA) (NatureWorks, USA) is fabricated by compression molding PLA pellets into the PDMS mold at 180° C. under vacuum for 2 hours. The PLA mold is then transferred onto a glass slide. This second mold, which has smaller dimensions and the same relative spacing, is aligned using a custom-built alignment device, then pressed into the needles which had been heated prior to this process. At an elevated temperature, the second mold would penetrate into the polymer, creating dimple-like structures. After peeling off the second mold, a two-dimensional array of hollow conical microneedle outer layer embedded inside the PDMS mold is obtained.

Step 2. Fabrication of exenatide-filled microneedle inner layer. A second negative PDMS mold having identical two-dimensional array of conical cavities, but with a smaller cone dimensions than the first negative mole in Step 1, is prepared by the same procedure described in Step 1. A two-dimensional PLGA film is then compression molded and impinged with a positive mold to give a two-dimensional array of conical hollow microneedles. The scum layer is then removed by high velocity spinning accompanied with addition of water or organic solvent such as acetone. A mixture of acetyl-exenatide trifluoroacetate (0.5 mg) in 1 mL of purified water and 0.1 to 1.0 mM aqueous solution of a suitable dye absorbing or emitting in the visible (400-650 nm) or NIR (650-900 nm) range are prepared and diluted to the final desired concentrations, and the entire mixture is solution-casted onto to the PDMS mold and allowed to dry. The scum layer is removed spinning and addition of distilled water. The scum-free drug-filled inner layer is then transferred onto a sacrificial layer comprising a solid polymer film of PLGA, placed on top of the mold, and compressed at the glass transition temperature under vacuum. The entire drug-filled inner layer is delaminated onto a solid substrate, such as a glass slide, using heat-assisted micro-transfer molding. The exenatide- and dye-filled inner layer is then aligned with and immersed into the outer layer in Step 1 resulting in the attachment of the two layers.

Step 3. Fabrication and attachment of the cap-layer to the core-shell. The cap layer and supporting array for the core-shell microneedles are fabricated using the same procedure in Step 1. A polymer film of PLGA or PLA is compression molded into PDMS mold of the caps and supporting array, respectively. The supporting array is transferred to a glass substrate and further coated with a water-soluble polymer solution of PVP K30 in ethanol (0.2 g/ml), then air-dried for 24 hours. The two-dimensional cap layer and the supporting layer are aligned with the inner and outer layers in Step 2 and sintered using heat. The entire exenatide-microneedle drug-devices system (patch) is detached (peeled off) from the mold. The resulting system is then used as the patch for transdermal delivery of exenatide.

### Example 2 (Prophetic)

### Semaglutide Core-Shell Microneedle Patch System

Step 1. Fabrication of PDMS mold. The Semaglutide powder can be obtained from Adipogen Pharmaceutical, USA. Poly lactic-co-glycolic acid (PLGA) can be obtained from Polysciences inc., USA. PVP can be obtained from the Thermo Fisher Scientific Inc., USA. Semaglutide Elisa assay kit can be obtained from Creative Diagnostic Inc., USA. The master structures of core, shell, array, and cap (available from Nano fiber facility) are replicated by curing PDMS on wafers. The mixture of the PDMS base and curing agent (Sylgard 184, Dow Corning) at a ratio of 7:1 is poured onto the wafers and then degassed under a vacuum for at least 1 h before curing in the room temperature for overnight. The PDMS molds are then gently peeled off from the silicon wafers and used for later fabrication process.

Step 2. Fabrication of PVP core and scum removal process. Semaglutide (6 mg) is dissolved into 600 μL of 5 M PVP solution (PVP, K30; Millipore Sigma) and 0.1 to 1.0 mM aqueous solution of a pyrazine dye with the wavelength of absorption or emission at about 450-650 nm range are prepared and diluted to the final desired concentrations. The entire mixture is then casted onto the PDMS mold and placed under vacuum for removal of solvent. After drying, the scum of the PVP on the mold is removed by using water as a solvent. The sacrificial layer is a solid polymer film of PLGA that is placed on top of the mold and then compressed at its glass transition temperature under vacuum. This whole structure is delaminated onto a solid substrate, such as a glass slide, using heat-assisted micro transfer molding.

Step 3. Fabrication of shell microneedles. The shell of the MNs is fabricated by using different kind of PLGA into 700 μm Shell molds and superficial layer of PLGA is removed by using acetone. PVP core were aligned into Shell by heating method.

Step 4. Fabrication of microneedle caps and supporting array. The supporting array for the core-shell microneedles are fabricated following similar procedures of compression molding and scum removal. The supporting array is fabricated from water-soluble polymer solution of PVP K30 in ethanol (0.2 g/mL), sodium bicarbonate, and citric acid mixture. PLA is used as a backing layer for array. The caps that were made from PLGA and trapped inside the PDMS mold are aligned with the PVP-coated PLA supporting array and sintered using heat. These MNs are further processed for various other analysis. In next step, effervescent array are attached to core-shell to form the complete MNs patch.

### Example 3 (Prophetic)

### Liraglutide Core-Shell Microneedle Patch System

Microneedle patch is fabricated in a nearly identical manner as described in Example 1, except that in Step 3, the inner layer is filled with liraglutide.

### Example 4 (Prophetic)

### Dulaglutide Core-Shell Microneedle Patch System

Microneedle patch is fabricated in a nearly identical manner as described in Example 1, except that in Step 3, the inner layer is filled with dulaglutide.

### Example 5 (Prophetic)

### Amylin Core-Shell Microneedle Patch System

Microneedle patch is fabricated in a nearly identical manner as described in Example 1, except that in Step 3, the inner layer is filled with Amylin.

### Example 6 (Prophetic)

### GIP Core-Shell Microneedle Patch System

Microneedle patch is fabricated in a nearly identical manner as described in Example 1, except that in Step 3, the inner layer is filled with GIP.

### Example 7 (Prophetic)

### Leptin Core-Shell Microneedle Patch System

Microneedle patch is fabricated in a nearly identical manner as described in Example 1, except that in Step 3, the inner layer is filled with leptin.

### Example 8 (Prophetic)

### Ghrelin Core-Shell Microneedle Patch System

Microneedle patch is fabricated in a nearly identical manner as described in Example 1, except that in Step 3, the inner layer is filled with ghrelin.

### Example 9 (Prophetic)

### CCK Core-Shell Microneedle Patch System

Microneedle patch is fabricated in a nearly identical manner as described in Example 1, except that in Step 3, the inner layer is filled with CCK.

### Example 10 (Prophetic)

### OXM Core-Shell Microneedle Patch System

Microneedle patch is fabricated in a nearly identical manner as described in Example 1, except that in Step 3, the inner layer is filled with OXM.

### Example 11 (Prophetic)

### PYY Core-Shell Microneedle Patch System

Microneedle patch is fabricated in a nearly identical manner as described in Example 1, except that in Step 3, the inner layer is filled with PYY.

### Example 12 (Prophetic)

### PP Core-Shell Microneedle Patch System

Microneedle patch is fabricated in a nearly identical manner as described in Example 1, except that in Step 3, the inner layer is filled with PP.

### Example 13 (Prophetic)

### Exenatide-GIP Core-Shell Microneedle Patch System

Microneedle patch is fabricated in a nearly identical manner as described in Example 1, except that in Step 3, the inner layer is filled with exenatide and GIP.

### Example 14 (Prophetic)

### Exenatide-Amylin Core-Shell Microneedle Patch System

Microneedle patch is fabricated in a nearly identical manner as described in Example 1, except that in Step 3, the inner layer is filled with exenatide and amylin.

### Example 15 (Prophetic)

### Exenatide-Leptin Core-Shell Microneedle Patch System

Microneedle patch is fabricated in a nearly identical manner as described in Example 1, except that in Step 3, the inner layer is filled with exenatide and leptin.

### Example 16 (Prophetic)

### Liraglutide-GIP Core-Shell Microneedle Patch System

Microneedle patch is fabricated in a nearly identical manner as described in Example 1, except that in Step 3, the inner layer is filled with liraglutide and GIP.

### Example 17 (Prophetic)

### Liraglutide-Amylin Core-Shell Microneedle Patch System

Microneedle patch is fabricated in a nearly identical manner as described in Example 1, except that in Step 3, the inner layer is filled with liraglutide and amylin.

### Example 18 (Prophetic)

### Liraglutide-Leptin Core-Shell Microneedle Patch System

Microneedle patch is fabricated in a nearly identical manner as described in Example 1, except that in Step 3, the inner layer is filled with liraglutide and leptin.

### Example 19 (Prophetic)

### Semaglutide-GIP Core-Shell Microneedle Patch System

Microneedle patch is fabricated in a nearly identical manner as described in Example 1, except that in Step 3, the inner layer is filled with semaglutide and GIP.

### Example 20 (Prophetic)

### Semaglutide-Amylin Core-Shell Microneedle Patch System

Microneedle patch is fabricated in a nearly identical manner as described in Example 1, except that in Step 3, the inner layer is filled with semaglutide and amylin.

### Example 21 (Prophetic)

### Semaglutide-Leptin Core-Shell Microneedle Patch System

Microneedle patch is fabricated in a nearly identical manner as described in Example 1, except that in Step 3, the inner layer is filled with semaglutide and leptin.

### Example 22 (Prophetic)

### Dulaglutide-GIP Core-Shell Microneedle Patch System

Microneedle patch is fabricated in a nearly identical manner as described in Example 1, except that in Step 3, the inner layer is filled with dulaglutide and GIP.

### Example 23 (Prophetic)

### Dulaglutide-Amylin Core-Shell Microneedle Patch System

Microneedle patch is fabricated in a nearly identical manner as described in Example 1, except that in Step 3, the inner layer is filled with dulaglutide and amylin.

### Example 24 (Prophetic)

### Dulaglutide-Leptin Core-Shell Microneedle Patch System

Microneedle patch is fabricated in a nearly identical manner as described in Example 1, except that in Step 3, the inner layer is filled with dulaglutide and leptin.

### Example 25 (Prophetic)

### Exenatide-Ghrelin Core-Shell Microneedle Patch System

Microneedle patch is fabricated in a nearly identical manner as described in Example 1, except that in Step 3, the inner layer is filled with exenatide and ghrelin.

### Example 26 (Prophetic)

### Exenatide-CCK Core-Shell Microneedle Patch System

Microneedle patch is fabricated in a nearly identical manner as described in Example 1, except that in Step 3, the inner layer is filled with exenatide and CCK.

### Example 27 (Prophetic)

### Exenatide-OXM Core-Shell Microneedle Patch System

Microneedle patch is fabricated in a nearly identical manner as described in Example 1, except that in Step 3, the inner layer is filled with exenatide and OXM.

### Example 28 (Prophetic)

### Exenatide-PYY Core-Shell Microneedle Patch System

Microneedle patch is fabricated in a nearly identical manner as described in Example 1, except that in Step 3, the inner layer is filled with exenatide and PYY.

### Example 29 (Prophetic)

### Exenatide-PP Core-Shell Microneedle Patch System

Microneedle patch is fabricated in a nearly identical manner as described in Example 1, except that in Step 3, the inner layer is filled with exenatide and PP.

### Example 30 (Prophetic)

### Semaglutide-Ghrelin Core-Shell Microneedle Patch System

Microneedle patch is fabricated in a nearly identical manner as described in Example 1, except that in Step 3, the inner layer is filled with semaglutide and ghrelin.

### Example 31 (Prophetic)

### Semaglutide-CCK Core-Shell Microneedle Patch System

Microneedle patch is fabricated in a nearly identical manner as described in Example 1, except that in Step 3, the inner layer is filled with semaglutide and CCK.

### Example 32 (Prophetic)

### Semaglutide-OXM Core-Shell Microneedle Patch System

Microneedle patch is fabricated in a nearly identical manner as described in Example 1, except that in Step 3, the inner layer is filled with semaglutide and OXM.

### Example 33 (Prophetic)

### Semaglutide-PYY Core-Shell Microneedle Patch System

Microneedle patch is fabricated in a nearly identical manner as described in Example 1, except that in Step 3, the inner layer is filled with semaglutide and PYY.

### Example 34 (Prophetic)

### Semaglutide-PP Core-Shell Microneedle Patch System

Microneedle patch is fabricated in a nearly identical manner as described in Example 1, except that in Step 3, the inner layer is filled with semaglutide and PP.

### Example 35 (Prophetic)

### Liraglutide-Ghrelin Core-Shell Microneedle Patch System

Microneedle patch is fabricated in a nearly identical manner as described in Example 1, except that in Step 3, the inner layer is filled with liraglutide and ghrelin.

### Example 36 (Prophetic)

### Liraglutide-CCK Core-Shell Microneedle Patch System

Microneedle patch is fabricated in a nearly identical manner as described in Example 1, except that in Step 3, the inner layer is filled with liraglutide and CCK.

### Example 37 (Prophetic)

### Liraglutide-OXM Core-Shell Microneedle Patch System

Microneedle patch is fabricated in a nearly identical manner as described in Example 1, except that in Step 3, the inner layer is filled with liraglutide and OXM.

### Example 38 (Prophetic)

### Liraglutide-PYY Core-Shell Microneedle Patch System

Microneedle patch is fabricated in a nearly identical manner as described in Example 1, except that in Step 3, the inner layer is filled with liraglutide and PYY.

### Example 39 (Prophetic)

### Liraglutide-PP Core-Shell Microneedle Patch System

Microneedle patch is fabricated in a nearly identical manner as described in Example 1, except that in Step 3, the inner layer is filled with liraglutide and PP.

### Example 40 (Prophetic)

### Dulaglutide-Ghrelin Core-Shell Microneedle Patch System

Microneedle patch is fabricated in a nearly identical manner as described in Example 1, except that in Step 3, the inner layer is filled with dulaglutide and ghrelin.

### Example 41 (Prophetic)

### Dulaglutide-CCK Core-Shell Microneedle Patch System

Microneedle patch is fabricated in a nearly identical manner as described in Example 1, except that in Step 3, the inner layer is filled with dulaglutide and CCK.

### Example 42 (Prophetic)

### Dulaglutide-OXM Core-Shell Microneedle Patch System

Microneedle patch is fabricated in a nearly identical manner as described in Example 1, except that in Step 3, the inner layer is filled with dulaglutide and OXM.

### Example 43 (Prophetic)

### Dulaglutide-PYY Core-Shell Microneedle Patch System

Microneedle patch is fabricated in a nearly identical manner as described in Example 1, except that in Step 3, the inner layer is filled with dulaglutide and PYY.

### Example 44 (Prophetic)

### Dulaglutide-PP Core-Shell Microneedle Patch System

Microneedle patch is fabricated in a nearly identical manner as described in Example 1, except that in Step 3, the inner layer is filled with dulaglutide and PP.

### REFERENCES

1. Runge S.; et al. Crystal structure of the ligand-bound glucagon-like peptide-1 receptor extracellular domain. The Journal of Biological Chemistry 2008, 283, 1134-11347.
2. Samms, R et al. How may GIP enhance the efficacy of GLP-1? Trends in Endocrinology & Metabolism 2020, 31, 410-421.
3. Caruso, I. et al.; Heterogeneity and similarities in GLP-1receptor agonist cardiovascular outcomes trials. Trends in Endocrinology & Metabolism 2019, 30, 578-589.
4. Nauck, M. A.; Quast, D. R.; Wefers, J.; Meier, J. J. GLP-1 receptor agonists in the treatment of type 2 diabetes-state of the art. Molecular Metabolism 2021, 46, 1-26.
5. Parker, V. E. R. et al. Efficacy, safety, and mechanistic insights of cotadutide, a dual-receptor glucagon-like peptide-1 agonist. Journal of Clinical Endocrinology and Metabolism 2020, 105, 803-820.
6. Lau, D. C. W et al. Once weekly Cagrilintide for weight management in people with overweigh and obesity: a multicentre, randomized, double-blind, placebo-controlled, and active-controlled, dose-finding phase 2 trial. The Lancet, volume 398, issue10317, P2160-2172 (2021)
7. Becerril. S et al. Cagrilintide plus semaglutide for obesity management. The Lancet 2021, 397, 1687-1689.
8. J. J. Host et al. GIP as a therapeutic target in diabetes and obesity: Insight from Incretin Co-agonists. Journal of Clinical Endocrinol and Metabolism 2020, 105, 105.
9. Killion. E. A et al. Anti-obesity effects of GIPR antagonists alone and in combination with GLP-1R agonists in preclinical models. Sci.Transl.Med. 2018, 10, 10.
10. Lu et al. GIPR antagonist antibodies conjugated to GLP-1 peptide are bispecific molecules that decrease weight in obese mice and monkeys. Cell Rep. Med. 2021, 2, 100263-1-100263-17.
11. Chodorge, M et al. Engineering of a GLP-1 analogue peptide/anti-PCSK9 antibody fusion for type 2 diabetes treatment. Sci. Rep. 2018, 8, 17545.
12. Marso et al. SUSTAIN TRIAL. New England Journal of Medicine 2016, 375, 1834-1844.
13. Gerstein et al. REWIND TRIAL. The Lancet 2019, 394, 121-130.
14. Gerstein et al. Dulaglutide and renal outcomes in type 2 diabetes. Lancet 2019, 394 (10193), 131-138.
15. Bajaj et al. Erectile function in men with type diabetes treated with dulaglutide. Lancet Diabetes and Endocrinology 2021, 9, 484-490.
16. Smith, S. R. et al. Sustained weight loss following 12-month pramlintide treatment as an adjunct to lifestyle intervention in obesity. Diabetes Care 2008, 31, 1816-1823.
17. Liberini, C. G. et al. Combined amylin/GLP-1 pharmacotherapy to promote and sustain long-lasting weight loss. Scientific Reports 2019, 9:8447, 1-11.
18. Kanoski, S. E. et al. Liraglutide, leptin and their combined effects on feeding: additive intake reduction through common intracellular signaling mechanisms. Diabetes, Obesity and Metabolism 2014, 30, 578-589.
19. Ambrey, P. et al. MEDI0382, a GLP-1 and glucagon receptor dual agonist, in obese or overweight patients with type-2 diabetes: a randomised, controlled, double-blind, ascending dose and phase 2a study. The Lancet 2018, http://dx.doi.org/10.1016/S0140-6736(18)30726-8.
20. Cappozzi, M. E. et al. Targeting the incretin/glucagon system with triagonists to treat diabetes. Endocrine Reviews 2018, 39, 719-738.
21. Hartman et al. Effects of Novel Dual GIP and GLP-1Receptor Agonist Tirzepatide on Biomarkers of Nonalcoholic Steatohepatitis in patients with type 2 diabetes. Diabetes Care 2020, 43, 1352-1355.
22. Soudy, R. et al. Short amylin antagonist receptor peptides improve memory deficits in Alzheimer's disease mouse model. Scientific Reports 2019, 9:10942, http://dx.doi.org/10.1038/S41598-019-47225-9.
23. McLean, P. L. et al. Lixisenatide, a drug developed to treat type 2 diabetes, shows neuroprotective effects in a mouse model of Alzheimer's disease. Neuropharmacology 2014, 86, 241-258.
24. Grizzanti et al. Journal of Alzheimer's Disease 2018, 66 (1): 11-23.
25. McHugh, K. J.; Guarecuco, R.; Langer, R.; Jaklenec, A. Journal of Controlled Release 2015, 219, 596-60.
26. Bajaj et al. Switching to once-weekly insulin Icodec versus once-daily insulin glargine U100 in type 2 diabetes inadequately controlled on daily basal insulin: A phase 2 randomized controlled trial. Diabetes care 2021; 44, 1586-1594.
27. Zhou et al. Microneedle-based insulin delivery system: current status and translational challenges. Drug Delivery and Translational Research 2022, 12, 2403-2427.
28. Nguyen, T. et al. Core-shell microneedle platform for transdermal and pulsatile drug/vaccine delivery and method of manufacturing the same. U.S. Patent Publication 2019, 2019/0269895.
29. Thanh, T. M. et al. Transdermal microneedles for the programmable burst release of multiple vaccine payloads. Nature Biomedical Engineering 2020, https://doi.org/10.1038/s41551-020-006504).
30. McHugh, K. J.; Nguyen, T. D.; Linehan, A. R.; Yang, D.; Behrens, A. M.; Rose, S.; Tochka, Z. L.; Tzeng, S. Y.; Norman, J. J.; Anselmo, A. C., Fabrication of fillable microparticles and other complex 3D microstructures. Science 2017, 357 (6356), 1138-1142.
31. Adams T. B. et al. The FEMA GRAS assessment of pyrazine derivatives as flavor ingredients. Food and Chemical Toxicology 2002, 40, 429-451.
32. Shirai, K. et al. Synthesis and fluorescent properties of 2,5-diamino-3,6-dicyanopyrazines dyes. Dyes and Pigments 1998, 39, 49-68.
33. Rajagopalan, R. et al. Hydrophilic pyrazine dyes as exogenous fluorescent tracer agents for real-time point-of-care measurement of glomerular filtration rate. Journal of Medicinal Chemistry 2011, 54, 5048-5058.
34. Rajagopalan, R. et al. Pyrazine Derivatives and Uses Thereof in Renal Function Monitoring. U.S. Pat. No. 8,115,000, 2012.
35. Rajagopalan, R. et al. Pyrazine Derivatives for Bioconjugation. U.S. Pat. No. 8,664,392, 2014.
36. Dorshow, R. B. et al. Pyrazine Derivatives and Uses Thereof, Including in Medical Imaging and Visualization Applications. U.S. Pat. No. 9,283,288, 2016.

We claim:

1. A programmable drug-device microneedle patch system for transdermal pulsatile delivery and optical monitoring of medications for the treatment of diabetes, obesity, and associated conditions, said microneedle system comprising:
(a) a biodegradable polymeric outer layer embedded with a two-dimensional array of conical hollow microneedles with the base diameter ranging from about 100 μm to about 500 μm and the height ranging from about 100 μm about 1200 μm that adheres to and fully encapsulates an inner layer, wherein the biodegradable polymer comprises PGA, PLA, or PLGA;
(b) a biodegradable polymeric inner layer embedded with a two-dimensional array of conical drug and optical dye containing microneedles with the base diameter ranging from about 100 μm to about 500 μm and the height ranging from about 100 μm about 1200 μm, wherein the drug is at least one peptide hormone selected from the group consisting of insulin, GLP-1RA, GIP, amylin, ghrelin, leptin, CCK, OXM, PYY, PP, GIPR antibody, and a GLP-1/PCSK9 inhibitor; the optical dye absorbs and emits light in the range of 400-900 nm; the biodegradable polymer comprises PGA, PLA, or PLGA; and the inner layer adheres to and is fully encapsulated by the outer layer;
(c) the inner layer is optionally further loaded with an effective amount of at least one peptide hormone selected from the group consisting of insulin, GLP-1R agonist, GIP, amylin, cagrilintide, amylin analog, ghrelin, leptin, CCK, OXM, PYY, and PP; and
(d) a biodegradable polymeric cap layer embedded with a two-dimensional array of cylindrical discs with the base diameter ranging from about 100 μm to about 500 μm and the height ranging from about 100 μm about 500 μm that aligns with and adheres to the basal surface of said inner and outer layers, wherein the biodegradable polymer comprises PGA, PLA, or PLGA.

2. The microneedle system of claim 1, wherein the optical dye is a pyrazine molecule represented by structural Formula 1, 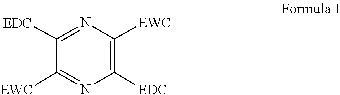
wherein EDG is —NR¹R²; EWG is —CN, —CONR³R⁴, or —CO₂R⁵; each of R¹ to R⁴ is independently selected from the group consisting of hydrogen, C₁-C₁₀ alkyl, C₂-C₁₂ mono or polyhydroxyalkyl, C₂-C₁₂ mono or polycarboxyalkyl, or —(CH₂CH₂O)ₙR⁶; R⁶ is hydrogen or methyl; and the subscript ‘n’ varies from 1 to 1000.

3. The microneedle system of claim 2, wherein the dye is a pyrazine molecule represented by structural Formula 1, wherein EDG is —NR¹R²; EWG is —CN, or —CONR³R⁴;
each of R¹ to R⁴ is independently selected from the group consisting of hydrogen, C₁-C₆ alkyl or —(CH₂CH₂O)ₙR⁶;
R⁶ is hydrogen or methyl; and
the subscript ‘n’ varies from 1 to 100.

4. The microneedle system of claim 2, wherein the dye is a pyrazine molecule represented by structural Formula 1, wherein
EDG is —NR¹R²; EWG is —CN, or —CONR³R⁴;
each of R¹ and R² is independently hydrogen or methyl;
each of R³ and R⁴ is independently hydrogen or —(CH₂CH₂O)ₙR⁶;
R⁶ is hydrogen or methyl; and
the subscript ‘n’ varies from 1 to 50.

5. The microneedle system of claim 1, wherein said inner layer is loaded with GLP-1RA.

6. The microneedle system of claim 2, wherein the GLP-1RA is exenatide, liraglutide, semaglutide, dulaglutide, lixisenatide, or albiglutide.

7. The microneedle system of claim 3, wherein the GLP-1RA is semaglutide, dulaglutide, or albiglutide.

8. The microneedle system of claim 3, wherein the GLP-1RA is semaglutide.

9. The microneedle system of claim 1, wherein said inner layer is loaded with insulin, amylin, cagrilintide, pramlintide, or GIP.

10. The microneedle system of claim 1, wherein said inner layer is loaded with ghrelin, leptin, CCK, OXM, PYY, or PP.

11. The microneedle system of claim 1, wherein said inner layer is loaded with two peptide hormones: semaglutide and glucagon, semaglutide and amylin, semaglutide and cagrilintide, semaglutide and pramlintide, or semaglutide and GIP.

12. The microneedle system of claim 1, wherein said inner layer is loaded with two peptide hormones: dulaglutide and glucagon, dulaglutide and amylin, dulaglutide and cagrilintide, dulaglutide and pramlintide, or dulaglutide and GIP.

13. The microneedle system of claim 1, wherein said inner layer is loaded with two peptide hormones: albiglutide and glucagon, albiglutide and amylin, albiglutide and cagrilintide, albiglutide and pramlintide, or albiglutide and GIP.

14. The microneedle system of claim 1, wherein said inner layer is loaded with two peptide hormones: insulin and GLP-1RA.

15. The microneedle system of claim 10, wherein the GLP-1RA is semaglutide, dulaglutide, or albiglutide.

16. The microneedle system of claim 1, wherein said inner layer is loaded with three peptide hormones: semaglutide, GIP, and glucagon.

17. The microneedle system of claim 1, wherein said inner layer is loaded with three peptide hormones: dulaglutide, GIP, and glucagon.

18. The microneedle system of claim 1, wherein said inner layer is loaded with three peptide hormones: albiglutide, GIP, and glucagon.

19. The microneedle system of claim 1, wherein said inner layer is loaded with insulin and an anti-obesity medication selected from the group comprising GIP, amylin, cagrilintide, pramlintide, ghrelin, leptin, CCK, OXM, PYY, or PP.

20. The microneedle system of claim 16, wherein the anti-obesity medication is GIP, amylin, or cagrilintide.

21. The microneedle system of claim 1, wherein said inner layer is loaded with GIPR antibody.

22. The microneedle system of claim 1, wherein said inner layer is loaded with GLP-1/PCSK9 inhibitor.

23. The microneedle system of claim 1, wherein said inner layer is loaded with GLP-1RA and GIPR antibody.
